(19) United States
(12) Patent Application Publication
(10) Pub. No.: US 2024/0341584 A1
(43) Pub. Date: Oct. 17, 2024

(54) COLLAPSIBLE DENTAL SCOPE

(71) Applicant: GET-GRIN INC., Airmont, NY (US)

(72) Inventor: Carmi RAZ, Gizo (IL)

(21) Appl. No.: 18/751,908

(22) Filed: Jun. 24, 2024

Related U.S. Application Data
(63) Continuation of application No. PCT/US23/10355, filed on Jan. 6, 2023.
(60) Provisional application No. 63/297,768, filed on Jan. 9, 2022.

(51) Int. Cl. A61B 1/247; A61B 90/00; A61C 19/00

(52) U.S. Cl. CPC A61B 1/247; A61B 90/361; A61C 19/00

(57) ABSTRACT

Described herein is an intraoral adaptor comprising a collapsible body, wherein the collapsible body comprises (i) a proximal end configured to couple the intraoral adapter to at least a portion of a subject's mouth, (ii) a distal end, and (iii) a viewing channel located between said proximal end and said distal end, wherein the viewing channel is configured to define a field of view of an intraoral region of the subject's mouth, wherein the collapsible body is configured to transform between an extended position and a collapsed position along an axis extending from the proximal end of the collapsible body to the distal end of the collapsible body.

### CROSS REFERENCE TO RELATED APPLICATIONS

This application is a Continuation of International Patent Application PCT/US23/10355, filed Jan. 6, 2023, which claims benefit of U.S. Provisional Application No. 63/297,768, filed Jan. 9, 2022, each of which is incorporated herein by reference in its entirety.

### TECHNICAL FIELD

The present invention, in at least some embodiments, relates to systems and methods for dental assessment, including remote dental assessment, and more specifically to systems and methods using a collapsible intraoral adaptor.

### BACKGROUND

Dental professionals may treat and monitor a patient's dental condition based on in-person visits. Treatment and monitoring of a patient's dental condition may require a patient to schedule multiple in-person visits to a dentist. The quality of treatment and the accuracy of monitoring may vary depending on how often and how consistently a patient sees a dentist. In some cases, suboptimal treatment outcomes may result if a patient is unable or unwilling to schedule regular visits to a dentist.

### SUMMARY

Recognized herein is a need for improved remote dental monitoring solutions to allow dental patients to receive high quality dental care, without requiring a dental professional to be physically present with the patient. Some dental professionals may use conventional teledentistry solutions to accommodate patients' needs and schedules. However, such conventional teledentistry solutions may provide inadequate levels of supervision. Further, such conventional teledentistry solutions may be limited by an inaccurate or insufficient monitoring of a patient's dental condition based on one or more photos or videos taken by a user if the photos do not adequately capture various intraoral features.

The present disclosure, according to at least some embodiments, provides systems and methods comprising a collapsible intraoral adaptor for use in remote dental assessment or monitoring. As used herein, the term “remote dental assessment or monitoring” may refer to assessment or monitoring conducted by a dental professional at a location which is remote from that of the user and may refer to the acquisition of one or more intraoral videos and/or intraoral images that can be assessed using a remote computing device, or can be sent to a different remote location for assessment.

The systems and methods disclosed herein may provide a convenient solution and user experience for dental patients to capture one or more intraoral videos or images using a mobile device such as a smartphone. The methods and systems disclosed herein may provide dentists with a detailed analysis of the patient's dental condition based images or videos of at least a portion of the intraoral region captured remotely by a user.

The present disclosure provides systems and methods, in some embodiments comprising a collapsible intraoral adaptor, that can be provided to the user or a patient for self-assembly prior to use. The collapsible dental adaptor of the present invention provides a dental device which is easily portable and requires less space for storage and transportation as compared to a non-collapsible device of similar dimensions.

In an aspect, provided herein is an intraoral adaptor comprising a collapsible body, wherein the collapsible body comprises (i) a proximal end configured to couple the intraoral adapter to at least a portion of a subject's mouth, (ii) a distal end, and (iii) a viewing channel located between said proximal end and said distal end, wherein the viewing channel is configured to define a field of view of an intraoral region of the subject's mouth, wherein the collapsible body is configured to transform between an extended position and a collapsed position along an axis extending from the proximal end of the collapsible body to the distal end of the collapsible body.

In some cases, a distance between the proximal end and the distal end of the collapsible body is at least 45 millimeters when the collapsible body is in the extended position.

In some cases, a distance between the proximal end and the distal end of the collapsible body is at most45 millimeters when the collapsible body is in the collapsed position.

In some cases, the collapsible body comprises at least a first portion and a second portion, wherein the second portion is configured to retract into the first portion when the collapsible body is in the collapsed position.

In some cases, when the collapsible body is in the collapsed position, the length of the collapsible body is equal to the length of the first portion.

In some cases, a width of the viewing channel of the collapsible body along the first portion is greater than a width of the viewing channel of the collapsible body along the second position.

In some cases, a proximal end of the second portion is wider than a distal end of the first portion, such that the distal end of the first portion is configured to be inserted into the proximal end of the second portion.

In some cases, when the collapsible body is in the extended position, an inner surface of the second portion overlaps an outer surface of the first portion, and wherein friction between the outer surface of the first portion and the inner surface of the second portion provides resistance against the transformation of the collapsible body between the extended position and the collapsed position.

In some cases, the overlap between the inner surface of the second portion and the outer surface of the first portion prevents entrance of external light into the viewing channel between the first portion and the second portion.

In some cases, the distal end of the first portion is connected to the proximal end of the second potion by a flexible material configured to allow nesting of the second portion within the first portion.

In some cases, the flexible material prevents entrance of external light into the viewing channel between the first portion and the second portion.

In some cases, the collapsible body is configured to transform between the collapsed position to the extended position.

In some cases, the proximal end further comprises an element that is configured to couple the intraoral adaptor to at least a portion of the subject's mouth.

In some cases, the element comprises a rim that extends outwardly from at least a portion of the proximal end of the collapsible body.

In some cases, the element is configured to be positioned outside of the viewing channel.

In some cases, the distal end comprises a mounting mechanism, wherein the mounting mechanism is configured to reversibly couple the intraoral adaptor to a mobile device comprising a camera.

In some cases, the mounting mechanism comprises a first attachment element for reversible coupling to the mobile device.

In some cases, the mobile device comprises a second attachment element, and wherein the first attachment element is configured to reversibly couple to the second attachment element.

In some cases, the first attachment element and the second attachment element each comprise at least one magnet.

In some cases, the first attachment element and the second attachment element each comprise Velcro.

In some cases, the mounting mechanism comprises a flexible band configured to retain the mobile device.

In some cases, the first attachment element comprises at least one hook shaped flange and the second attachment element comprises at least one loop, wherein the hook shaped flange is configured to be insertable into the loop.

In some cases, the first attachment element or the second attachment element comprises a multi-use sticker.

In some cases, the mobile device further comprises a case or cover, and wherein the second attachment element is located on the case or cover.

In some cases, the first attachment element comprises at least two arms separated by a distance, wherein the distance can be adjusted to secure the mobile device between the at least two arms.

In some cases, each of the at least two arms can be adjusted independently.

In some cases, the intraoral adaptor further comprises a locking mechanism to lock the collapsible body in the extended position.

In another aspect, provided herein is a method for biometric identification or biometric authentication, comprising: (a) acquiring one or more videos or images of an intraoral region of a subject, wherein the one or more videos or images comprises at least a portion of an oral landmark of the subject; (b) comparing the one or more videos or images comprising at least a portion of the oral landmark of the subject with reference dental data, wherein the reference dental data is associated with a target subject; and (c) identifying a degree of similarity between the oral landmark of the subject acquired in the one or more videos or images and the reference dental data, wherein if the degree of similarity is equal to or above a threshold value, the subject is confirmed to be the target subject.

In some cases, the acquiring comprises providing a mobile device comprising a camera and acquiring the one or more videos or images using the camera.

In some cases, the mobile device is a mobile phone.

In some cases, an intraoral adaptor is coupled to the mobile device.

In some cases, the intraoral adaptor comprises an elongated housing comprising a proximal end configured to couple the intraoral adapter to at least a portion of a subject's mouth, a distal end, and a viewing channel extending between the proximal end and distal end.

In some cases, the intraoral adaptor further comprises a mounting mechanism provided at the distal end of the elongated housing, wherein the mounting mechanism is configured to reversibly couple the intraoral adaptor to a mobile device comprising a camera, and wherein when the intraoral adaptor is coupled to the mobile device, the viewing channel is configured to define a field of view of the intraoral region of the subject for image or video capture using the camera of the mobile device.

In some cases, the oral landmark comprises at least a portion of a dental arch.

In some cases, acquiring the one or more videos or images is performed under visible light illumination.

In some cases, acquiring the one or more videos or images is performed under UV light illumination.

In some cases, acquiring the one or more videos or images is performed under IR light illumination.

In some cases, the reference dental data comprises a set of coordinates relating to at least a portion of the oral landmark of the target subject.

In some cases, the reference dental data relates to a physical appearance of at least a portion of the oral landmark of the target subject.

In some cases, the method further comprises providing a 3D digital model of at least a portion of the intraoral region of the subject.

In some cases, the method further comprising converting the reference dental data into a set of coordinates representing a physical appearance of at least a portion of the intraoral region of the target subject.

In some cases, the method further comprising converting the reference dental data into a set of coordinates representing a physical appearance of at least a portion of the oral landmark of the target subject.

In some cases, the method further comprises converting the reference dental data into a 3D digital model representing a physical appearance of at least a portion of the intraoral region of the target subject.

In some cases, the comparing is performed using software and/or hardware of a mobile device.

In some cases, the comparing is performed using software and/or hardware of a remote computing device.

In some cases, the comparing comprises comparing at least one feature selected from the group consisting of outline and contours of one or more teeth, shapes and sizes of one or more teeth, distances and position of one or more teeth, appearance and shape of at least one dental arch, 3D structure of one or more teeth, evidence of dental and intraoral treatments or any visible intervention, defects of one or more teeth, intrinsic color of teeth, and stains present one or more teeth.

In some cases, the threshold value of similarity is 90%. In some cases, the threshold value of similarity is about 80% to about 99%. In some cases, the threshold value of similarity is about 80% to about 85%, about 80% to about 90%, about 80% to about 95%, about 80% to about 99%, about 85% to about 90%, about 85% to about 95%, about 85% to about 99%, about 90% to about 95%, about 90% to about 99%, or about 95% to about 99%. In some cases, the threshold value of similarity is about 80%, about 85%, about 90%, about 95%, or about 99%. In some cases, the threshold value of similarity is at least about 80%, about 85%, about 90%, or about 95%.

In some cases, if the subject is confirmed to be the target subject, the subject is authenticated to perform an action.

In some cases, the authentication comprises 3rd party authorization.

In some cases, the 3rd party authorization further requires at least one additional biometric marker and/or character identification of the subject.

In an aspect, provided herein is a method of confirming the identity of a subject, comprising: providing an intraoral adaptor according to claim 1; providing a mobile phone comprising a camera and a processor; using the intraoral adaptor, acquiring one or more videos or images of an intraoral region of the subject, wherein the one or more videos or images comprises at least a portion of an oral landmark of the subject; comparing the one or more videos or images comprising at least a portion of the oral landmark of the subject with reference dental data, wherein the reference dental data is associated with a target subject; and identifying a degree of similarity between the oral landmark of the subject acquired in the one or more videos or images and the reference dental data, wherein if the degree of similarity is equal to or above a threshold value, the subject is confirmed to be the target subject. when the identified degree of correspondence is at least 90%, providing the identification verification or authorization verification.

In some cases, the threshold value of similarity is 90%.

In some cases, the oral landmark relates to a medical condition or physical appearance of at least a portion of the intraoral region of the subject.

In some cases, the oral landmark is selected from the group consisting of a tooth, a gingiva, a tongue, an uvula, an airway, a gland or an intraoral tissue.

In some cases, if the subject is confirmed to be the target subject, the subject is authenticated to perform an action.

In some cases, the action comprises signing a document.

In some cases, the action comprises providing authorization.

In some cases, if the subject is confirmed to be the target subject, the subject is able to obtain access to a secured physical or virtual location or to secured physical or virtual assets.

In some cases, if the subject is confirmed to be the target subject, a transaction is confirmed.

In some cases, if the subject is confirmed to be the target subject, the subject gains access to an on-line banking site.

In another aspect, provided herein is a method for assessing a condition of at least a portion of an intraoral region of a remote subject, comprising: providing an intraoral adaptor according to claim 1; providing a mobile phone comprising a camera and a processor; acquiring from the subject one or more images or videos of the intraoral region using the intraoral adaptor coupled to the mobile phone; and analyzing at least one oral landmark of the subject present in the one or more images or videos.

In some cases, the at least one oral landmark relates to a medical condition or physical appearance of at least a portion of the intraoral region of the subject.

In some cases, the portion of the intraoral region is selected from the group consisting of a tooth, a gingiva, a tongue, an uvula, an airway, a gland and an intraoral tissue. In some cases, the method is used for remote assessment.

In some cases, the assessment is provided prior to, during, or subsequent to a medical or cosmetic treatment.

In some cases, the method is used for 3D mapping of the intraoral cavity or teeth of the subject.

Unless otherwise defined, all technical and scientific terms used herein have the same meaning as commonly understood by one of ordinary skill in the art to which this invention belongs. Although suitable methods and materials are described below; methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the method and system of the present invention involves performing or completing selected tasks or steps manually, automatically, or a combination thereof. Moreover, according to actual instrumentation and equipment of preferred embodiments of methods, apparatus and systems of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIGS. 1A-1C schematically illustrate different views of an example of a collapsible intraoral adaptor, in accordance with the principles of the present invention.

FIGS. 2A-2D schematically illustrate different views a further example of a collapsible intraoral adaptor, in accordance with the principles of the present invention.

FIGS. 3A-3E schematically illustrate different views of the collapsible intraoral adaptor of FIGS. 1A-1C coupled to a mobile phone.

FIG. 4 schematically illustrates a computer system that is programmed or otherwise configured to implement at least some of the methods disclosed herein, in accordance with the principles of some embodiments of the present invention.

FIG. 5 and FIG. 6 are flow-charts schematically illustrating example methods of dental assessment, in accordance with the principles of some embodiments of the present invention.

FIG. 7 is a flow chart schematically illustrating a method for assessing a condition of an intraoral region of a remote subject.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

As used herein, when a numerical value is preceded by the term “about”, the term “about” is intended to indicate +/−10% of that value. Whenever the term “at least,” “greater than,” or “greater than or equal to” precedes the first numerical value in a series of two or more numerical values, the term “at least.” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “a.” “an,” and “the,” as used herein, generally refer to singular and plural references unless the context clearly dictates otherwise.

### Overview

According to an aspect, the present disclosure provides systems and methods for intraoral assessment. As used herein, “intraoral assessment” may refer to assessment of an appearance or condition of an intraoral region of a subject. The methods and systems disclosed herein may provide a convenient solution and user experience for dental patients to capture one or more intraoral videos or images using a mobile device such as a smartphone. The methods and systems disclosed herein may provide dentists and orthodontists with a detailed analysis of the patient's dental condition based on a video or on one or more images of at least one tooth, such as one or more teeth, one or more dental arches or both dental arches of a subject, captured remotely by a user. In some embodiments, the user is the subject. In some embodiments the user is a person other than the subject, present at the same location as the subject and assessment of the captured video or image(s) is performed at a remote location.

As used herein the term “dental scope” or “dental adaptor” refer to a device that can be attached to a camera or mobile phone and enables capturing at least one intraoral image or videos of a subject. An example and possible uses can be seen in U.S. patent application Ser. No. 17/336,997. The systems and methods of the present disclosure may be implemented using a software application that is configured to enable a dental patient to capture videos and/or images of intraoral regions. The software application may be used by a user or a subject (e.g., a dental patient) in conjunction with a mobile device to remotely monitor a dental condition of the subject. The dental condition may comprise a development, a growth, a movement, an appearance, a condition, a physical arrangement, a position, and/or an orientation of the subject's teeth.

The intraoral adaptor may be used to enable remote monitoring. As used herein, “remote monitoring” may refer to monitoring a condition of an intraoral region of a subject, wherein the monitoring is performed at one or more locations remote from the subject. For example, a dentist or a medical specialist may monitor the intraoral anatomy or intraoral condition at a first location that is different than a second location at which the subject is located. The first location and the second location may be separated by a distance spanning at least 1 meter, 1 kilometer, 10 kilometers, 100 kilometers, 1000 kilometers, or more.

The remote monitoring may be performed by assessing an intraoral condition of the subject using one or more dental scans captured from the subject when the subject is located remotely from the dentist or a dental office. In some cases, the remote monitoring may be performed in real-time such that a dentist is able to assess the dental condition when a subject uses a mobile device to acquire one or more videos or images of one or more intraoral regions in the patient's mouth. The remote monitoring may be performed using equipment, hardware, and/or software that is not physically located at a dental office.

### Device for Intraoral Assessment

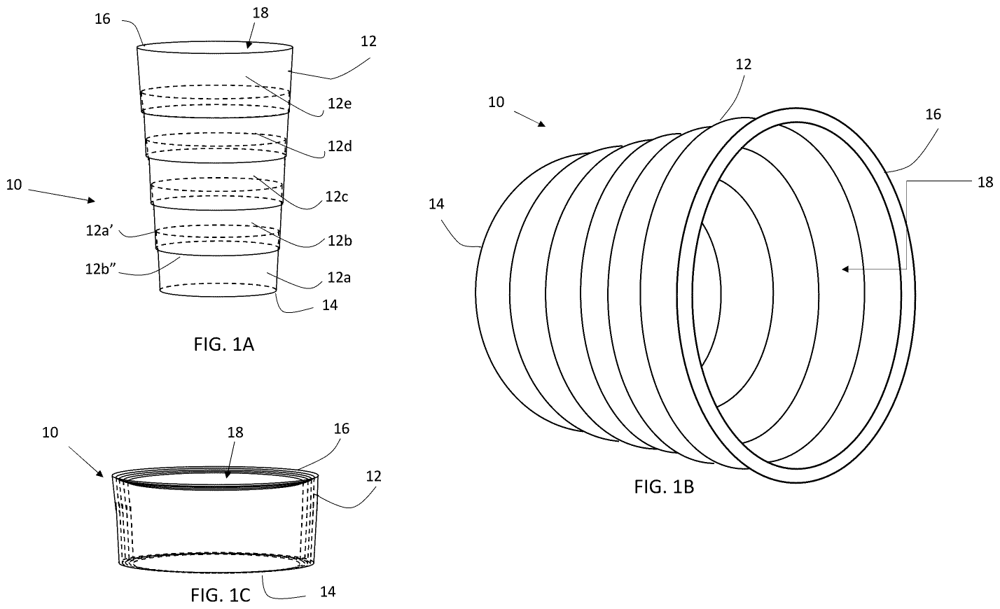

FIGS. 1A-1C schematically illustrate an example of an embodiment of a collapsible intraoral adaptor 10, in accordance with the principles of the present invention.

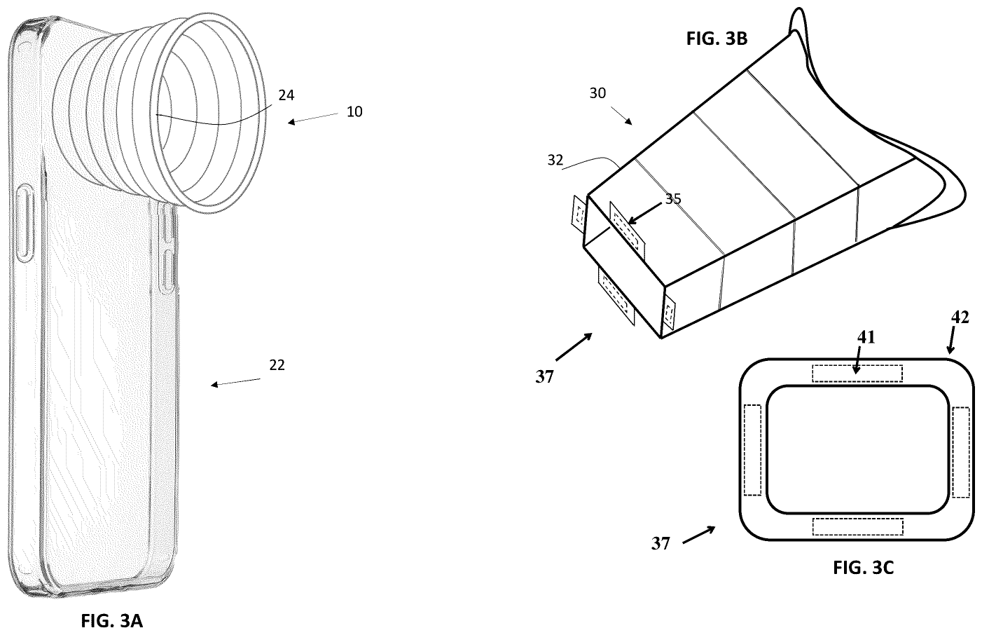

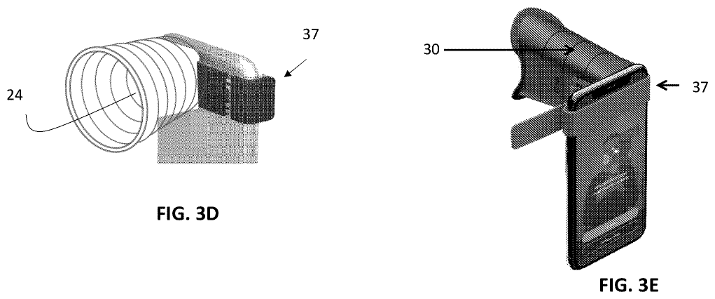

Intraoral adaptor 10 may comprise: a body 12 having an open proximal end 14, an open distal end 16 configured for insertion within an intraoral region of a subject, and a viewing channel 18 extending between proximal end 14 and distal end 16; and a mounting mechanism 20 (shown in FIGS. 3A-3E) provided at proximal end 14.

Mounting mechanism 20 can be configured to reversibly couple intraoral adaptor 10 to a mobile device 22 (shown in FIGS. 3A-3E) comprising a camera 24. Body 12 can be configured to be collapsible from an extended state (as shown in FIG. 1A. 1B) to a collapsed state (as shown in FIG. 1C) along an axis (not shown) connecting proximal end 14 to distal end 16. When intraoral adaptor 10 is coupled to mobile device 22, viewing channel 18 can be configured to define a field of view of an intraoral region of a subject for capture of at least one image or video using camera 24 of mobile device 22.

A distance between proximal end 14 and distal end 16 can be at least about 45 millimeters when body 12 is in the extended state. In an extended position, the distance between the proximal end can be about 45 mm to about 100 mm. In an extended position, the distance between the proximal end can be about 45 mm to about 50 mm, about 45 mm to about 75 mm, about 45 mm to about 100 mm, about 50 mm to about 75 mm, about 50 mm to about 100 mm, or about 75 mm to about 100 mm. In an extended position, the distance between the proximal end can be about 45 mm, about 50 mm, about 75 mm, or about 100) mm. In an extended position, the distance between the proximal end can be at least about 45 mm, about 50 mm, or about 75 mm. In an extended position, the distance between the proximal end can be at most about 50 mm, about 75 mm, or about 100 mm.

A distance between proximal end 14 and distal end 16 can be at most 45 millimeters when body 12 is in the collapsed state. In a collapsed position, the distance between the proximal end can be about 10 mm to about 45 mm. In a collapsed position, the distance between the proximal end can be about 10 mm to about 25 mm, about 10 mm to about 40) mm, about 10 mm to about 45 mm, about 25 mm to about 40) mm, about 25 mm to about 45 mm, or about 40 mm to about 45 mm. In a collapsed position, the distance between the proximal end can be about 10 mm, about 25 mm, about 40 mm, or about 45 mm. In a collapsed position, the distance between the proximal end can be at least about 10 mm, about 25 mm, or about 40 mm. In a collapsed position, the distance between the proximal end can be at most about 25 mm, about 40 mm, or about 45 mm.

Body 12 may comprise five concentric elements or sections 12a, 12b, 12c, 12d, 12e, each having a circular cross-section, wherein first element 12a is telescopically collapsible or retractable into second element 12b, second element 12b is telescopically collapsible or retractable into third element 12c, third element 12c is telescopically collapsible or retractable into fourth element 12d, and fourth element 12d is telescopically collapsible or retractable into fifth element 12e. For example, in some cases, distal end 12a′ of first element 12a has a peripheral dimension which is less than a peripheral dimension of a proximal end 12b″ of second element 12b, such that proximal end 12b″ of second element is insertable within distal end 12a′ of first element 12a, and so on. Body 12 is collapsible to the height of first element 12a.

The peripheral dimension of a portion of an outer surface of first element 12a may substantially correspond to a peripheral dimension of a portion of an inner surface of second element 12b, such that when body 12 is in the extended state, a portion of the outer surface of first element 12a overlaps with a portion of the inner surface of second element 12b (as shown in FIG. 1A). In some cases, frictional resistance between the portion of the outer surface of element 12a and the portion of the inner surface of second element 12b provides physical resistance to collapse.

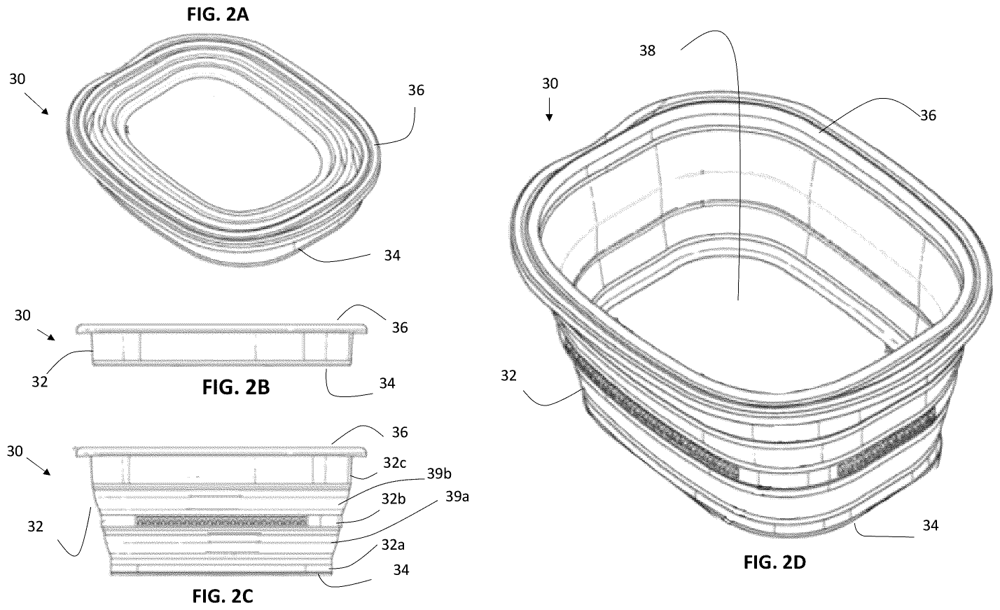

FIGS. 2A-2D schematically illustrate an example of a collapsible intraoral device 30, in accordance with the principles of the present invention.

Intraoral adaptor 30 may comprise: a body 32 having an open proximal end 34, an open distal end 36 configured for insertion within an intraoral region of a subject, and a viewing channel 38 extending between proximal end 34 and distal end 36; and a mounting mechanism 37 (show in FIGS. 3A-3E) provided at proximal end 34.

Mounting mechanism 37 can be configured to reversibly couple intraoral adaptor 30 to a mobile device 22 (shown in FIGS. 3A-3E) comprising a camera 24. Body 32 can be configured to be collapsible from an extended state (as shown in FIG. 2C. 2D) to a collapsed state (as shown in FIG. 2A. 2B) along an axis (not shown) connecting proximal end 34 to distal end 36. When intraoral adaptor 30 is coupled to mobile device 22, viewing channel 38 can be configured to define a field of view of an intraoral region of a subject for capture of at least one image or video using camera 24 of mobile device 22.

A distance between proximal end 34 and distal end 36 is at least about 45 millimeters when body 32 is in the extended state. In an extended position, the distance between the proximal end can be about 45 mm to about 100 mm. In an extended position, the distance between the proximal end can be about 45 mm to about 50 mm, about 45 mm to about 75 mm, about 45 mm to about 100 mm, about 50 mm to about 75 mm, about 50 mm to about 100 mm, or about 75 mm to about 100 mm. In an extended position, the distance between the proximal end can be about 45 mm, about 50 mm, about 75 mm, or about 100 mm. In an extended position, the distance between the proximal end can be at least about 45 mm, about 50) mm, or about 75 mm. In an extended position, the distance between the proximal end can be at most about 50 mm, about 75 mm, or about 100 mm.

A distance between proximal end 34 and distal end 36 is no greater than 45 millimeters when body 32 is in the collapsed state. In a collapsed position, the distance between the proximal end can be about 10 mm to about 45 mm. In a collapsed position, the distance between the proximal end can be about 10 mm to about 25 mm, about 10 mm to about 40) mm, about 10) mm to about 45 mm, about 25 mm to about 40 mm, about 25 mm to about 45 mm, or about 40 mm to about 45 mm. In a collapsed position, the distance between the proximal end can be about 10 mm, about 25 mm, about 40) mm, or about 45 mm. In a collapsed position, the distance between the proximal end can be at least about 10 mm, about 25 mm, or about 40 mm. In a collapsed position, the distance between the proximal end can be at most about 25 mm, about 40 mm, or about 45 mm.

Body 32 may comprise 3 concentric elements or sections 32a, 32b, 32c, wherein first element 32a is telescopically collapsible or retractable into second element 32b, and second element 32b is telescopically collapsible or retractable into third element 32c. For example, in some cases, distal end 32a′ of first element 32a has a peripheral dimension which is less than a peripheral dimension of a proximal end 32b″ of second element 32b, such that proximal end 32b″ of second element is insertable within distal end 32a′ of first element 32a, and so on. Body 32 can be collapsible to the height of first element 32a.

First element 32a can be connected to second element 32b by a flexible material 39a, configured to allow nesting of second element 32b within first element 32a. Similarly, second element 32b can be connected to third element 32c by a flexible material 39b, configured to allow nesting of third element 32c within second element 32b.

In some embodiments, the elongated housing further comprises an element that is configured (i.e. sized and shaped) to couple the intraoral adaptor to at least a portion of the subject's mouth.

In some such embodiments, the element further comprises at least one outwardly extending rim around at least a portion of the proximal end of the elongated housing.

According to some such embodiments, the intraoral adaptor is coupled to a portion of the intraoral region between an outer surface of the teeth and an inner surface of the lips.

In some embodiments, the element that is configured to couple the intraoral adaptor to the subject's mouth is further configured to be positioned outside the viewing channel.

In some embodiments, the mounting mechanism comprises an attachment element for reversible coupling to the mobile device.

In some embodiments, the mobile phone comprises an attachment element and the attachment element of the elongated housing is configured for reversible coupling to the attachment element of the mobile device.

In some embodiments, the attachment element of the elongated housing and the attachment element of the mobile device each comprise at least one magnet (such as one, two, three, four or more than four magnets).

In some embodiments, the attachment element of the elongated housing and the attachment element of the mobile device each comprise g Velcro.

In some embodiments, the mounting mechanism comprises a flexible band configured to retain the mobile device. The band may comprise any material having sufficient flexibility to stretch around a perimeter of the mobile device and sufficient strength to retain the device therein.

In some embodiments, the attachment element of the elongated housing comprises at least one hook shaped flange and the attachment element of the mobile device comprises at least one loop, wherein the hook shaped flange is configured to be insertable into the loop. In some embodiments, the attachment element of the mobile device comprises at least one hook shaped flange and the attachment element of the elongated housing comprises at least one loop, wherein the hook shaped flange is configured to be insertable into the loop

In some embodiments, the attachment element of the elongated housing and/or the attachment element of the mobile device comprises at least one (such as one, two, three, four or more than four) multi-use sticker. In some embodiments, the attachment element of the elongated housing comprises at least one (such as one, two, three, four, or more than four) multi-use sticker. In some embodiments, the attachment element of the mobile device comprises at least one (such as one, two, three, four, or more than four) multi-use sticker. In some embodiments, each of the attachment element of the elongated housing and the attachment element of the mobile device comprises at least one (such as one, two, three, four, or more than four) multi-use sticker.

In some embodiments, the mobile device further comprises a case or cover, wherein the attachment element of the mobile device is provided on the case or cover.

In some embodiments, the attachment element of the elongated housing comprises at least two spaced-apart extendable arms, configured, when extended, to hold therebetween the mobile device.

In some embodiments, each of the at least two extendable arms adaptor is independently extendable.

In some embodiments, the intraoral adaptor further comprises a locking mechanism to lock the elongated housing in the extended position. In some such embodiments, the locking mechanism is selected from the group consisting of a rotating bar, a sliding bar, a pin, a clip and the like.

FIG. 3A schematically illustrates the collapsible intraoral device 10 of FIGS. 1A-1C coupled to a mobile device comprising a mobile phone 22 having a camera 24.

FIG. 3B schematically illustrates a mounting mechanism 37 comprising a plurality of magnets35 for coupling intraoral device 30 of FIGS. 2A-2D coupled to a mobile device comprising a mobile phone 22 having a camera 24. Alternatively or additionally, mounting mechanism 35 may comprise at least one adhesive strip, hook or Velcro, or a combination thereof.

FIG. 3C schematically illustrates a mounting mechanism 37 comprising a frame 42 on which are provided a plurality of magnets 41 for coupling intraoral device 30 of FIGS. 2A-2D coupled to a mobile device comprising a mobile phone 22 having a camera 24. Alternatively or additionally, frame 42 of mounting mechanism 35 may comprise at least one adhesive strip, loops for hooks or Velcro, or a combination thereof.

FIGS. 3D and 3E schematically illustrate the collapsible intraoral device 10 of FIGS. 1A-1C coupled to a mobile device comprising a mobile phone 22 having a camera 24 by a mounting mechanism 37 comprising a flexible band.

### Method for Biometric Identification or Biometric Authentication

### Computer Systems

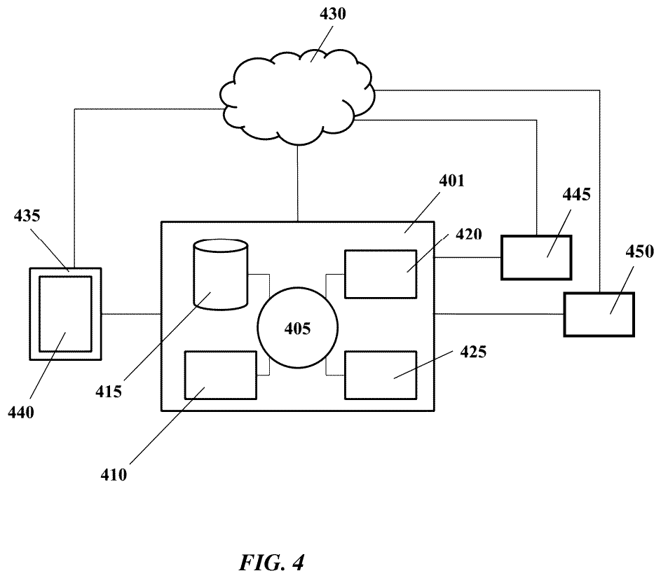

FIG. 4 shows a computer system 401 that is programmed or otherwise configured to implement a method for dental assessment. The computer system 401 may be configured to, for example, process intraoral videos or images captured using the camera of the mobile device, and determine at least one of the dental assessment properties during the dental assessment. The computer system 401 can be an electronic device of a user or a computer system that is remotely located with respect to the electronic device. The electronic device can be a mobile electronic device. The computer system 401 can be a smartphone.

The computer system 401 may include a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk. Solid State drive or equivalent storge unit), communication interface 420) (e.g., network adaptor) for communicating with one or more other systems, and peripheral devices 425, such as cache, other memory, data storage and/or electronic display adaptors. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are located external to the computer system 401 (e.g., on a remote server that is in communication with the computer system 401 through an intranet or the Internet).

The computer system 401 can communicate with one or more remote computer systems through the network 430. For instance, the computer system 401 can communicate with a remote computer system of a user (e.g., a subject, a dental patient, or a dentist). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a storage unit. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM. DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a portal for a subject or a dental patient to view one or more intraoral images or videos captured using a mobile device of the subject or the dental patient. In some cases, the electronic display 435 may be the feedback element providing the generated output, for example displaying message or shape or light in accordance to some embodiments. The portal may be provided through an application programming interface (API). A user or entity can also interact with various elements in the portal via the UI. Examples of UI's include, without limitation, a graphical user interface (GUI) and web-based user interface.

The computer system 401 can include or be in communication with a Camera 445 for providing, for example, ability to capture videos or images of the user or a dental patient. And for example, retrieve at least one dental scan date (such as optical object distance) that can be used to analyzed and compered to at least one dental scan properties

Methods and systems of the present disclosure can be implemented by way of one or more algorithms. An algorithm can be implemented by way of software upon execution by the central processing unit 405. The algorithm can, for example, implement a method for dental scan. The method may comprise processing videos or images captured using the camera of the mobile device or processing dental assessment data sensed by at least one sensor that can be used to analyze and compare to at least one dental assessment properties and executed to generate output.

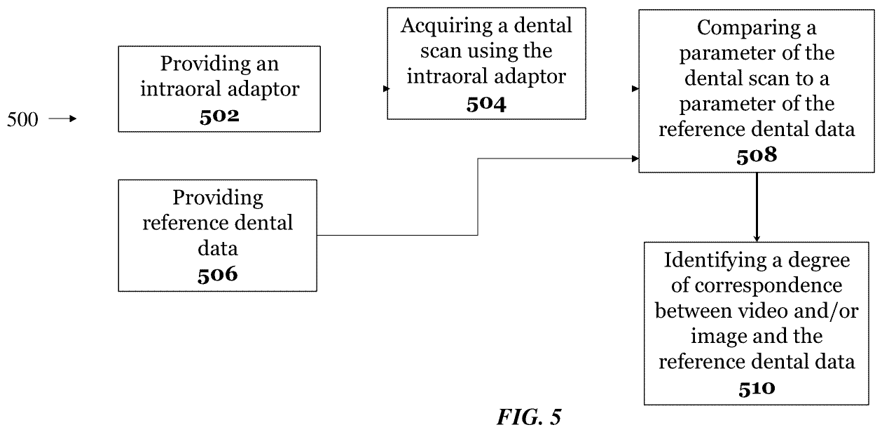

FIG. 5 is a flow-chart schematically illustrating an exemplary method for biometric identification or biometric authentication 500, in accordance with the principles of the present invention.

In some cases, method 500 comprises providing an intraoral adaptor comprising a body having an open proximal end and an open distal end and a viewing channel extending therebetween to a subject (step 502); acquiring a dental scan comprising at least one video and/or image of at least a portion of an intraoral region of a subject using the intraoral adaptor (step 504); providing reference dental data comprising at least one dental parameter of the subject (step 506); comparing the at least one parameter present in the video and/or image of the dental scan to at least one corresponding parameter present in the reference dental data (step 508); and identifying a degree of correspondence between the parameter of the dental scan and that of the reference dental data (step 510). The intraoral device is optionally substantially identical to the collapsible intraoral device as disclosed herein and represented in FIGS. 1A-1C, 2A-2D, or 3A-3E. Alternatively, the intraoral device may be different from the collapsible intraoral device as disclosed herein, such as, for example, that disclosed in U.S. patent application Ser. No. 17/336,997.

In some embodiments, acquiring the dental scan 504 comprises providing a mobile device (such as a mobile phone or electronic tablet) comprising a camera and acquiring the video and/or image using the camera. In some such embodiments, the portion of the intraoral region is viewed by the lens of the camera of the mobile device, positioned at the proximal end of the intraoral device, with the lens of the camera directed into the viewing channel.

In some embodiments, the method further comprises providing a source of light (such as visible light, UV light or IR light), wherein the step of acquiring the video and/or image is performed under illumination by the visible, UV or IR light.

In some embodiments, the comparing is performed using software and/or hardware of the mobile device and/or using software and/or hardware of a remote computing device.

In some embodiments, the comparing comprises comparing of at least one feature selected from the group consisting of outline and contours of teeth, shapes and sizes of teeth, distances and position of teeth, appearance and shape of at least one dental arch, 3D structure of teeth, evidence of dental and intraoral treatments or any visible intervention, defects of teeth, intrinsic teeth color and stains of teeth, and combinations thereof.

In some embodiments, wherein the identified degree of correspondence is at least about 90%, the method further comprises providing an identification confirmation or authentication confirmation.

In some embodiments, the confirmation comprises 3rd party authorization.

In some such embodiments, the 3rd party authorization further requires at least one additional biometric marker and/or character identification.

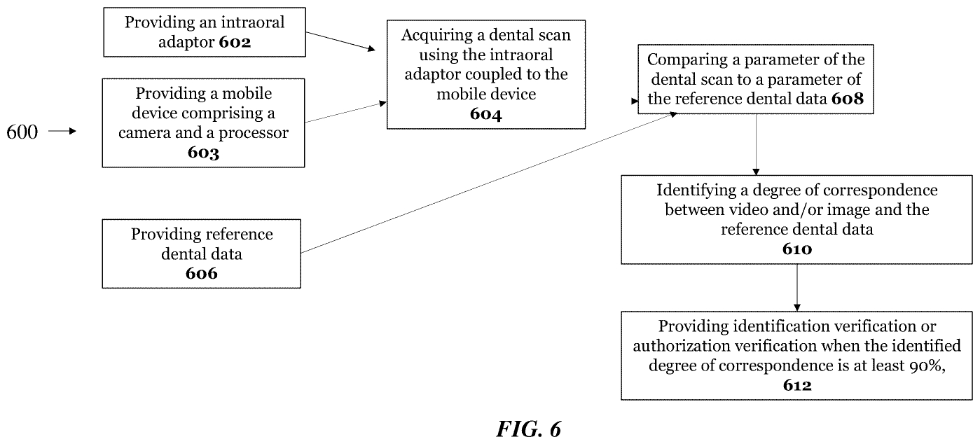

FIG. 6 is a flow-chart schematically illustrating an exemplary method 600 for obtaining an identification verification or authentication verification of a remote subject in accordance with the principles of the present invention.

In some cases, method 600 comprises, providing an intraoral adaptor as disclosed herein (step 602); providing a mobile device, such as a mobile phone, comprising a camera and a processor (step 603); acquiring from the subject a dental scan comprising at least one video and/or image of at least a portion of the intraoral region using the intraoral adaptor coupled to the mobile phone (step 604); providing reference dental data comprising at least one dental parameter of the subject (step 606); comparing a parameter of the dental scan to a corresponding parameter present in a reference dental data of the subject (step 608); identifying a degree of correspondence between the parameter present in the video and/or image and the parameter present in the reference dental data (step 610); and when the identified degree of correspondence is at least 90%, providing the identification verification or authorization verification (step 612).

According to some embodiments of the method, the at least one parameter relates to a medical condition or physical appearance of at least a portion of the intraoral region of the subject.

### Method for Assessing a Condition of an Intraoral Region of a Remote User

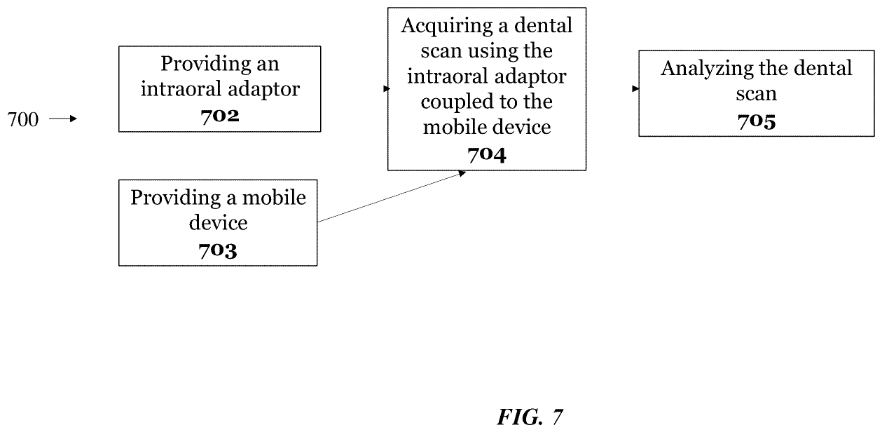

FIG. 7 schematically illustrates a method for assessing a condition of at least a portion of an intraoral region of a remote subject 700.

In some cases, method 700 comprises providing an intraoral adaptor as disclosed herein and shown in FIGS. 1A-1C. 2A-2D, or 3A-3E (step 702); providing a mobile device, such as a mobile phone, comprising a camera and a processor (step 703); acquiring from the subject a dental scan comprising at least one video and/or image of the intraoral region using the intraoral adaptor coupled to the mobile device (step 704); and analyzing at least one parameter of the image and/or video of the acquired dental scan obtained from the subject (step 705).

According to some embodiments, method 700 further comprises sending the acquired image to a remote location.

According to some embodiments, the at least one parameter relates to a medical condition or physical appearance of at least a portion of the intraoral region of the subject.

According to some embodiments of the method, the portion of the intraoral region is selected from the group consisting of a tooth, a gingiva, a tongue, an uvula, an airway, a gland and an intraoral tissue.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense.

Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An intraoral adaptor comprising a collapsible body, wherein the collapsible body comprises (i) a proximal end configured to couple the intraoral adapter to at least a portion of a subject's mouth, (ii) a distal end, and (iii) a viewing channel located between said proximal end and said distal end, wherein the viewing channel is configured to define a field of view of an intraoral region of the subject's mouth,
wherein the collapsible body is configured to transform between an extended position and a collapsed position along an axis extending from the proximal end of the collapsible body to the distal end of the collapsible body.

2. The intraoral adaptor according to claim 1, wherein the collapsible body comprises at least a first portion and a second portion, wherein the second portion is configured to retract into the first portion when the collapsible body is in the collapsed position.

3. The intraoral adaptor according to claim 2, wherein a proximal end of the second portion is wider than a distal end of the first portion, such that the distal end of the first portion is configured to be inserted into the proximal end of the second portion.

4. The intraoral adapter of claim 1, wherein the distal end comprises a mounting mechanism, wherein the mounting mechanism is configured to reversibly couple the intraoral adaptor to a mobile device comprising a camera.

5. A method for biometric identification or biometric authentication, comprising:
(a) acquiring one or more videos or images of an intraoral region of a subject, wherein the one or more videos or images comprises at least a portion of an oral landmark of the subject;
(b) comparing the one or more videos or images comprising at least a portion of the oral landmark of the subject with reference dental data, wherein the reference dental data is associated with a target subject; and
(c) identifying a degree of similarity between the oral landmark of the subject acquired in the one or more videos or images and the reference dental data, wherein if the degree of similarity is equal to or above a threshold value, the subject is confirmed to be the target subject.

6. The method of claim 5, wherein the acquiring comprises providing a mobile device comprising a camera and acquiring the one or more videos or images using the camera, wherein an intraoral adaptor is coupled to the mobile device.

7. The method of claim 6, wherein the mobile device is a mobile phone.

8. The method of claim 6, wherein the intraoral adaptor comprises an elongated housing comprising a proximal end configured to couple the intraoral adapter to at least a portion of a subject's mouth, a distal end, and a viewing channel extending between the proximal end and distal end.

9. The method of claim 8, wherein the intraoral adaptor further comprises a mounting mechanism provided at the distal end of the elongated housing,
wherein the mounting mechanism is configured to reversibly couple the intraoral adaptor to a mobile device comprising a camera, and
wherein when the intraoral adaptor is coupled to the mobile device, the viewing channel is configured to define a field of view of the intraoral region of the subject for image or video capture using the camera of the mobile device.

10. The method of claim 5, wherein the oral landmark comprises at least a portion of a dental arch.

11. The method of claim 5, wherein acquiring the one or more videos or images is performed under visible light illumination.

12. The method of claim 5, wherein acquiring the one or more videos or images is performed under UV light illumination.

13. The method of claim 5, wherein acquiring the one or more videos or images is performed under IR light illumination.

14. The method of claim 5, wherein the reference dental data comprises a set of coordinates relating to at least a portion of the oral landmark of the target subject.

15. The method of claim 5, wherein the reference dental data relates to a physical appearance of at least a portion of the oral landmark of the target subject.

16. The method of claim 5, further comprising providing a 3D digital model of at least a portion of the intraoral region of the subject.

17. The method of claim 5, wherein the method further comprising converting the reference dental data into a set of coordinates representing a physical appearance of at least a portion of the intraoral region of the target subject.

18. The method of claim 5, wherein the method further comprising converting the reference dental data into a set of coordinates representing a physical appearance of at least a portion of the oral landmark of the target subject.

19. The method of claim 5, wherein the comparing comprises comparing at least one feature selected from the group consisting of outline and contours of one or more teeth, shapes and sizes of one or more teeth, distances and position of one or more teeth, appearance and shape of at least one dental arch, 3D structure of one or more teeth, evidence of dental and intraoral treatments or any visible intervention, defects of one or more teeth, intrinsic color of teeth, and stains present one or more teeth.

20. A method for assessing a condition of at least a portion of an intraoral region of a remote subject, comprising:
providing an intraoral adaptor according to claim 1;
providing a mobile phone comprising a camera and a processor;
acquiring from the subject one or more images or videos of the intraoral region using the intraoral adaptor coupled to the mobile phone; and
analyzing at least one oral landmark of the subject present in the one or more images or videos.
